(19) United States
(12) Patent Application Publication
(10) Pub. No.: US 2008/0199489 A1
(43) Pub. Date: Aug. 21, 2008

(54) SKIN TREATMENT FORMULATIONS AND METHOD

(76) Inventor: Vincene M. Parrinello, San Diego, CA (US)

Correspondence Address: Mak Wisnosky, Suite 1407, 110 West C, San Diego, CA, 92101, US

(21) Appl. No.: 11/675,965

(22) Filed: Feb. 16, 2007

(51) Int. Cl. A61K 36/02; A61P 17/00

(52) U.S. Cl. 424/195.17

(57) ABSTRACT

A series of natural herbal-based ozonated formulations and treatment steps effective in the treatment and prevention of inflammatory skin conditions is described. The formulations provide a novel approach to selection and preparation of medicated formulations where a cocktail approach is appropriate and a logical series of delivery matrices and processes are used to deliver the active components to the affected region or zone of the anatomy.

### TECHNICAL FIELD

Embodiments of the invention relate to skin treatment formulations and procedures applicable to healing and restoration of skin tissue damaged by environment and disease.

### BACKGROUND OF THE INVENTION

Cancer accounts for about ⅕ of all deaths in the United States. More than 1 million people in the United States are diagnosed with cancer annually. Cancer is characterized by uncontrolled division of a population of cells. These cells may metastasize spreading the disease to other sites throughout the body. Cancer of the skin is the most common type of cancer and is often described by the type of cells affected. Basal cells are located in the lowest layer of the epidermis. Basal cell carcinoma is the most common type of Cancer. Squamous cells make up most of the epidermis. Squamous cell carcinoma is the second most common type of cancer. Cancer of epidermis layer of the skin is generally thought to be caused by prolonged exposure to the sun but may also occur where skin has suffered certain kinds of injury: burns, scars, long-standing sores, sites previously exposed to X-rays or certain chemicals. Chemicals such as formaldehyde-based DMDM Hydantoin, BHT, ethanolamines (MEA, DEA, TEA), cyanide, surfactants (Sulfates), parabens, butylene glycol and other petroleum-derived products have all been implicated as hazardous and causing cancer. Even so, they are ubiquitous as product additives and in our environment. It is difficult to avoid exposure. In addition, chronic inflammatory dermatoses are known to be correlated to the development of skin cancers. Acne, precancerous condition of actinic keratosis, hyper pigmentation, psoriasis, eczema, rosacea, and couperose have all been correlated with later formation of cancer. Treatments and cures for both cancer and inflammatory diseases of the skin often look to naturally occurring chemicals. The antioxidants naturally occurring in teas have been found useful for the treatment of cancer and other diseases. In vitro studies suggest the unique polysaccharides of Spirulina enhance cell nucleus enzyme activity and DNA repair synthesis. Extracts have also been shown to have antiviral activity. Extracts of Laminaria Digitata, a genus of brown seaweeds are known to not only contain a distribution of essential amino acids that may aid in penetration of topically applied nutrients, but also contain azulene which has been shown to have anti-inflammatory properties. Callophycus Serratus, red seaweed found in the coral reefs off the coast of Fiji is a primary anti-cancer ingredient in theses new formulations. Many other common plants and herb extracts have also been shown to have medicinal anti-inflammatory properties. Amongst these, some non-exhaustive and non-limiting examples are Gotu Kola, Gingko Biloba, Arbutin from tea, evening primrose, borage, lavender, St. John's wort, aloe vera, Indian Gooseberry (Emblica), Prickly Pear and slippery Elm.

Green and white tea extracts have been shown to contain quantities of anti-oxidant such as polyphenols, flavinoids and phenolic acids. The polyphenols from teas are catechins. The anti-cancer properties of these catechins are now being further studied in clinical trials. These antioxidants have been shown to have anti-mutagenic properties. The inventor has also found that effectiveness of the teas varies by the methods used to grow the teas and the details of harvesting. Teas that are grown organically have been observed to have higher catechin levels. Harvesting at the appropriate time in the growth cycle as well as harvesting at the optimum time of day also affects the catechin levels in the extracts. Extracts from algaes are known to be rich in phycocyanins and carotenoids. These compounds have been demonstrated to have antiviral and anticancer activities. Immuno-stimulatory and anti-inflammatory properties have been reported for various extracts of kelp. Plant extracts, such as Nopal are rich in B vitamins. Studies have indicated that such pantothenates related to B vitamins can accelerate cell growth and migration to promote healing.

Cholecalciferol, vitamin D3, also known as the sunshine vitamin, aids in tissue regeneration and healing. This ingredient when used with the seaweeds of the invented formulas surprisingly increased the ozone output from the ozonized formulations when tested with a commercial ozone sensor. For this reason it was determined that a certain amount of vitamin D3 increases the effectiveness of formulations containing seaweeds, especially those containing callophycus serratus.

Otto Warburg won Nobel Prize in 1931 for showing that cancer can thrive in anaerobic and acidic conditions. Subsequent research has shown that an oxygen rich environment with a pH above 7.0 is selectively deadly for cancer cells. When alkaline water or oxygen is taken in by cancer cells the pH level rises and the oxygen content of the cell also rises. When this happens the cancer cells die. Similarly an oxygenated environment is well known for the treatment of Acne and other inflammatory skin diseases. Use of Ozonized terpenes for the treatment of cancers is an active area of current research.

A major problem with preparation and application of these oxygenated agents is their short life time in an aqueous environment. Half-lifes of less than an hour are typical for peroxide or ozone derivatives. The inventor has discovered that the pH of the formulation as well as a new storage system both increase the effective shelf life of the formulations. Solution with a pH in the range of 7 to 9, preferably 8, have shelf lives that extend the benefits of the ozonation by 6 months over formulations not controlled within this optimum pH zone.

The inventor also made the surprising discovery that storage of the ozonated formulations within a glass container that is then stored within a marble or similar stone container significantly increased the effective life of the product as indicated by measured ozone concentration. Storage in typical plastic containers (Acrylic, polyethylene or PET) results in a measured decrease in ozone and a maximum shelf life of 6 months. Unopened formulations stored in glass were found to maintain an effective level of ozone for 15 months. After 15 months the ozone level was reduced by 5%. Once opened the loss of ozone was found to be 2 to 5% every 10 days. By accident the inventor put a formulation in a glass jar and then inserted the glass jar into a travertine marble container. The formulation was found to last significantly longer than those stored in glass alone. Unopened storage life was extended from 15 to 24 months as measured by ozone concentration. Concentrations were found to still be at 20% ozone content even after this extended storage. Opened containers were also found to benefit from storage in a stone container. Potency as measured by ozone concentrations was found to be maintained above 14% even after 16 months. The best mode storage for the product is use of a glass container that contains no more than a 6-month supply and storage of the glass container within a marble or stone jar both prior to use and after opening and resealing. Hydrolysis by formulated or ambient water also reduces the beneficial oxidative potency of many formulations. Formulations containing water will have reduced shelf lives. Formulations are required that both use pharmaceutically acceptable carriers, diluents and excipients and maintain the activity of the agents contained therein. For this reason a series of treatments each with a varying moisture content level have been found to be more effective than a single treatment.

Epidermal and dermal carcinoma are generally treated either with topical pharmaceuticals such as that sold under the trade name Efudex®, or applied liquid nitrogen and biopsied with excising the area of concern and the surrounding area—leaving a laceration from the excision. Basal cell and squamous cell carcinoma are ordinarily treated with topical pharmaceutical ointments or retinoid creams and/or sprayed with liquid nitrogen. The use of liquid nitrogen often results in the skin having redness, flakiness and when the treated area heals, there is residual redness and indentation of the skin showing that of a scar. However the majority of patients with tumors also have micro-metastases beyond the immediately affected areas. Topical application as contrasted to systemic application permits high local concentrations of the effective agents and avoids many adverse side effects. Creating a change in the pH and oxidative state of an affected area on the skin simply could not be accomplished systemically without significant side effects. The greatest problem in applying physiologically active agents topically is that the skin is such an effective barrier to penetration. The epidermis has an exterior layer of dead cells called the stratum corneum which is tightly compacted and oily and which provides an effective barrier against gaseous, solid or liquid chemical agents, whether used alone or in water or oil solutions. Creation of the local pH and oxidative conditions conducive to treatment for the inflammatory dermatoses requires wetting and penetration of this outer layer. If a physiologically active agent can penetrate the stratum corneum it can then be effective in treating conditions of the squamous, basal and other structures of the epidermis and dermis, and be supportive of the epidermal immune cells, the Langerhans and Merkel cells. There is therefore a need for topical formulations and methods for applying the formulations that can preserve the potency of the formulation through manufacture, shipment and storage. There is a need for formulations that can be topically applied and penetrate the stratum corneum to reach affected regions within the epidermis and dermis. There is a need for specific formulations that can deliver therapeutically effective amounts of ozonated and non-ozonated compounds that will regionally produce oxygen rich and neutral to basic pH conditions for the treatment of cancers, pre-cancerous tissues and generally inflammatory dermatoses including acne, psoriasis, eczema, couperose, melasma, and enhance healing and reduce residual scarring from disease and injury. There is a need for a treatment method that incorporates both hydrophilic and hydrophobic excipients to allow delivery of a range of pharmaceutically effective compounds, which when amounts are decreased can be effective cosmeceuticals to aid challenged epidermal skin cells.

### SUMMARY OF THE INVENTION

We describe skin care formulations and methods of use for the treatment of cancerous, pre-cancerous, acne, rosacea, eczema, psoriasis, melasma, couperose, and general inflammatory conditions of the skin. The formulations allow for extended shelf life for easily hydrolyzed or otherwise labile formulation components. The innovative combination of ingredients in these formulations has produced a stabilized oxygen and ozone benefit. The formulations in combination with the novel methods of application deliver therapeutically effective oxygenated compounds to affected regions within the structure of the skin. The procedures described have been shown to provide immediate short-term relief to inflammatory conditions and also provide long-term maintenance for healing and restoration of health to epidermal skin cells which then act to protect dermal cells and prevent further deterioration of cells in the dermis. The formulations have been devised through application and assessment on patients diagnosed with cancerous and pre-cancerous skin conditions. We have made the unanticipated discovery that a treatment that combines four distinct steps for cleansing, activation, entrapping and sealing, each using a cocktail of ingredients and including ozonation, results in significantly improved effectiveness in treatment of cancerous and pre-cancerous skin conditions. The cleansing step prepares the skin by pre-wetting and infusion of the skin with the ozonated water broth in the nanoparticle vapors from the nebulizing infuser such as that sold under the trade name Dermoxyzone™. The activation step activates the skin using an ozonated gel serum. The entrapping step comprises a powdered mask and a mixing serum for the mask stirred into the powder to create an active ozonated mask. The effectiveness of the mask is further enhanced during treatment with the ozone solution (25% ozone) directly applied over the mask as it sits on the skin. The final “retention” or “sealing in” steps, uses ozonated and otherwise sterilized ingredients provide improved short term efficacy in treatment of inflammatory related skin ailments including pre-cancerous lesions and also provide in combination with the other three steps, unexpected prevention of recurrence of the ailments. The treatments have been found to be sufficiently effective that treatment frequencies could be reduced to maintenance levels without recurrence of the ailments. All four formulations are prepared from known naturally occurring anti-cancer, anti-inflammatory, anti-bacterial and anti-fungal ingredients. Formulations may be oxygenated and purified through use of ozone generator and addition of hydrogen peroxide to the formulation. It is believed by the inventor that these novel formulations are effective because of the ultra-purity afforded by the ozonation and formulation, a newly invented cocktail of anti-inflammatory, anti-bacterial, anti-fungal and anti-cancer ingredients, combined with ozonation to produce newly invented oxygenated formulations applied through novel delivery of the ingredients into the appropriate dermal layer through the combination of a pre-wetting step and the nanoparticle delivery system. Each of the formulation steps provided a different matrix for extraction and application of the active ingredients. Multiple treatments with varying hydrophilicity have been discovered to deliver healing activity not previously observed. Varying the amount of butters and oils included controls the hydrophilicity of the formulations. Hyaluronic acid is also used as a humectant and to aid adsorption through the skin to deliver effective amount of the active components to the affected layers of the skin. Additionally it is believed that the formulation and the preparation of the formulation with the use of the ozone and hydrogen peroxide results in formulations that maintain their effectiveness over time. The use of these two agents at the optimized pH level preserves formulations of skincare and hair-care products without using detrimental or controversial chemical preservatives, such as parabens and formaldehyde releasing preservatives. Adjusting the pH has allowed for creating optimal levels of sustaining the ozone/oxygen benefits in the formulations to sustain release of the ozone into the epidermis to “smother” cancer cells with the oxygen and ozonated benefits.

### DETAILED DESCRIPTION

The following non-limiting examples were prepared for each of the formulations and treatment steps. The best mode treatment comprises a water broth cleansing and pre-wetting step, an activating serum step, a mask step to entrap active ingredients against the skin, and finally a sealing cream is applied. Treatments may use any single step or combination of steps. The selected treatments may also be combined with formulations from other inventors and commercial formulations to synergistically improve their performance. The best mode formulations used in each of the treatment steps follow. The formulae ingredients are divided into main categories, such as waters, teas, plant extracts, etc. Effective and novel formulas were discovered by selecting ingredients from each of these categories. The best mode uses all of the selected ingredients from each category in the proportions listed. The invention comprises the specific formulations and the treatment methods using the formulations. Both are described in detail in following sections.

### Formulations

### Water Broth

### Water

One gallon of natural spring mineral waters is measured out selected from waters originating in Fiji, Hawaii, Northern Alps of Italy, Canada, Colorado and the French Alps. The best mode formulation comprised 20% Fiji, 15% Hawaii, 20% Northern Alps of Italy, 20% Canadian, 15% Colorado and 10% French Alps. The water should be equivalent to those sold under the following brand names respectively Fiji Bottled Water®, Hawaiian Bottled Water®, Panna® or Pelligrino® bottled water from Italy, Ice Age® bottled water from British Columbia, Canada and Evian® bottled water from the French Alps.

### Teas

Selections of teas, preferably organic, are added to the specially selected blended mineral water. The teas are known to produce extracts rich in catechins that have been demonstrated to have anti-cancer activity in clinical trials. The best mode formulation used:

10 ounces of white tea stems and leafs,
2 ounces of green tea
2 ounces of white tea
8 ounces of South African Rooibos tea,
5 ounces of Oolong tea and

### Algae and Seaweeds

A selection of Algae and seaweeds are also added to the mix and steeped in the same manner as and with the teas. The Seaweeds are known to have anti-viral and anti cancer activity as well as a source for hyaluronic acid that is a natural, known moisture or water binding ingredient. Hyaluronic acid is also a significant component of the skin structure itself and has been shown to enhance absorption through the outer stratum corneum and into the dermal layers most affected by inflammatory diseases. The best mode formulation comprised:

4 ounces Spirulina Blue Green Algae (microcystis aeruginosa)
4 ounces Sea Kelp (Laminaria Hyperborean)
5 ounces Atlantic Sea Kelp (Luminaria Digitata)
5 ounces Bladderwrack (Fucus Vesiculosus)
7 ounces Red Seaweed (Callophycus Serratus) ounces Knotted wrack (Ascophyllum Nodosom)
3 ounces Brown Algae (Phaeophyta)
3 ounces Sea Parsley (Levisticum officinale)
2 ounces Seal Lettuce (Ulva)

### Herbs

A selection of plants and herbs are similarly extracted. The plants herbs, teas flowers, fruits and vegetables have been selected from a list of plants known individually for their anti-inflammatory and in some cases anti-cancer activity. The best mode formulation comprised:

2 ounces st john's wort
5 ounces Indian Gooseberry
2 ounces slippery elm
3 ounces corkwood tree
2 ounces chickweed
3 ounces Nopal
2 ounces Gotu Kola
2 ounces black willow
5 ounces olive leaf
5 ounces grape leaf
2 ounces Gingko Biloba
4 ounces rosemary
4 ounces usnea (moss or lichen)
4 ounces Siegesbeckia Orientalis
5 ounces rosehip seed
4 ounces flax seed
3 ounces white ginger
3 ounces Licorice Root
1 ounce White Birch Bark
2 ounces Burdock Root

### Flowers

A selection of flowers are also added to the extraction mix. The flower extracts are in general anti-inflammatory and the inventor has observed that they have the ability to lighten the discoloration of damaged skin. The best mode formulation comprised:
4 ounces Roman Chamomile
4 ounces hibiscus
4 ounces lavender
4 ounces borage
2 ounces camellia
4 ounces sunflower
2 ounces Calendula
3 ounces Elderberry Blossom
5 ounces lotus flower

### Fruits

The inventor has found that addition of fruit extracts to the formulation contribute through exfoliation of dead skin cells and increased adsorption of active ingredients. Some of the extracts also have known anti-inflammatory and anti-cancer properties. Use of the water broth in combination with the other formulations and steps described below provides means to deliver healing and potent anti-oxidant protection not just to the outer layers of the skin but also to the basal junction. Where it can be more effective in treating the discussed inflammations and can provide sub-epidermal protection that can work in conjunction with normal topically applied sunscreens. The best mode formula comprised:

4 ounces Apple pectin in juice from four types of apples, includes the skin and core.
2 ounces persimmon
5 ounces Grapes—white, red and purple—includes skins, seeds and pulp
2 ounces papaya
4 ounces Italian Melon
4 ounces pineapple
4 ounces pomegranate
2 ounces black cherry
5 ounces red raspberry
5 ounces blueberry
5 ounces Kiwano Melon
5 ounces cranberry
5 ounces Hawaiian noni
7 ounces Brazilian Acai berry
5 ounces pipoano melon
5 ounces starfruit
7 ounces Prickly Pear

### Vegetables

The inventor also added vegetables into the extraction mix. The selected vegetables are sources of carotenoids, tested to have demonstrated anti-cancer activity and B vitamins that aid in repairing damaged cells. The best mode formulation comprised:

4 ounces carrot
4 ounces spinach
4 ounces kale
4 ounces tomato
4 ounces chard

### Other Additives

A selection of anti-oxidant, whitening, preservative and sterilization ingredients are also used. The best mode formulation comprised:

3 ounces arbutin from tea
2 ounces Potassium Sorbate
2 ounces ozonized ester C and ascorbic acid
3 ounces Tocopheryl vitamine E and Citric Acid
1 ounce per gallon of 35% hydrogen peroxide.

The teas, fruits, flowers, herbs, algae and seaweeds and vegetables are steeped in the selected waters at 90 C for 20 minutes. The best mode formulation found that addition in the order of teas, fruits, flowers, herbs, algae and seaweeds, and lastly vegetables produced the optimum results. After steeping, the solution is filtered by passing through an unbleached paper filter to remove visible sediment. Filtering is repeated approximately 25 times until the solution is free of sediment as indicated by free flow through the paper filter without clogging and the solution is visibly clear. The “Other Additives” are then added. The solution is then treated for 25 minutes by diffusion of output from an ozone generator at 250 mg/hour ozone generation such as that sold under the brand name Nature Kleen Ozonator™ from Transformation Technologies™. The hydrogen peroxide is added last. The solution is mixed well, bottled and then frozen for storage in a freezer at approximately 0 F. Prior to use the solution is thawed, preferably slowly under refrigeration at approximately 35 F until used.

### Activating Serum

Although the causative science is not completely understood, the serum provides a different matrix environment for the active ingredients extracted from the various plant materials and also provides a different matrix for interaction with the skin. The ingredients of the activating serum, a gel consistency—due to large amounts of botanical melon-hyaluronic acid, aloe vera gel and other melon content is comprised of crucial organic, botanical moisture or water binding ingredients (the melons, seaweeds and aloe vera) that retain 1000 times their weight in the infused nanop article water broth solution being infused into the epidermis. The industry norm for hyaluronic acid moisture binding abilities of water that is not nanoparticle size, not ozonated/oxygenated and not vitamin enriched, as is the water broth, is 400 times their weight. The inventor found that the use nanoparticle delivery of the water broth and the activating serum as a conductive agent produced a water binding delivery system better able to penetrate the epidermis and produce more effective results in the treatment of various previously described skin conditions. More effective means more dramatic improvement in the conditions and longer lasting effect of the observed improvement.

The activating serum uses the water broth described above as an ingredient. Additional ingredients selected from the same categories and for the same reasons as described for the water broth are then added to make a serum. The serum uses commercially available liquid extracts of each of the ingredients. An effective serum has been made from selecting various combinations of subsets of ingredients selected from the following categories. The best mode formulation uses all of the listed ingredients in the proportions indicated.

### Teas

1 fluid ounce Emperor's White Tea extract
1 fluid ounce Rooibos Red Tea extract
3 fluid ounces Green tea extract
8 fluid ounces Arbutin from tea extract

### Algae and Seaweeds

7 fluid ounces Red Seaweed (Callophycus Serratus)
5 fluid ounces Atlantic Kelp (Laminaria Digitata)
2 fluid ounces Bladderwrack (Fucus vesiculosus)
2 fluid ounces Sea Kelp (Laminaria Hyperborea)

### Herbs

1 fluid ounce Duboisia Leaf extract
1 fluid ounce horse chestnut extract
3 fluid ounces Indian Gooseberry (Phyllanthus Emblica) extract
10 fluid ounces Aloe Vera gel
1 fluid ounce licorice root extract

### Butters and Oils

4 fluid ounces Argan Oil

### Flowers

2 fluid ounces Lotus Flower extract
2 fluid ounces Linden flower extract
2 fluid ounces Lichen flower extract
3 fluid ounces Sea Buckthorn seed (Sallow Thorn) extract
3 fluid ounces White Iris (Iris Germanica) extract
3 fluid ounces Comfrey leaf extract

### Fruits

5 fluid ounces Kiwano Melon extract
5 fluid ounces Brazilian Melon extract
3 fluid ounces Italian Melon extract
4 fluid ounces Acai fruit extract
5 fluid ounces Pipano Melon extract
5 fluid ounces Starfruit extract
3 fluid ounces Pomegranate extract

### Other Additives

10 fluid ounces the water broth
1 fluid ounce 35% hydrogen peroxide
1 fluid ounce vitamin B3
1 fluid ounce vitamin B5
2 fluid ounces Sodium Hyaluronate
3 fluid ounces Soy Lecithin
3 fluid ounces Tocopheryl Acetate
1 fluid ounces Glycerol
3 fluid ounces Glycerol Stearate
2 fluid ounces sclerotium gum
2 fluid ounces Xanthan gum
4 fluid ounces Hyalurase Botanical Hyaluronic acid

The water broth and the ingredients selected from the categories of teas, algae and seaweeds, plants, flowers and fruits are blended together. The “Other Additives” are then added, blended well and puréed in a food processor to a “tomato juice” consistency and strained through a very fine mesh culinary strainer. The mixture is then treated for 25 minutes by diffusion of output from an ozone generator at 250 mg/hour ozone generation. The hydrogen peroxide is added last. The solution is mixed well and bottled.

### Mask

The mask is used after the water broth cleansing and activating serum activation steps. As for the serum the inventor has observed a synergistic efficacy by using the combination of treatments. Also as for the serum the exact mechanism is yet to be understood, but the mask provides yet a third matrix for the preparation, storage and application of the active ingredients. As for the formulations previously described the mask is prepared from a selection of ingredients form each of the defined categories of teas, Algaes and seaweeds, herbal plant extracts and oils, flower extracts, fruit extracts, vegetable extracts, butters and oils, and finally other additives as indicated in the formulation lists below. The inventor observed that the amount of ozone released from ozonated formulations increased significantly with the addition of cholecalciferol, Vitamin D3. For this reason Cholecalsciferol was added to the mixing serum of the mask to enhance the release of ozone and provide a more effective oxidative environment during the use of the mask. The mask is prepared from freeze dried and powdered extracts of the indicated ingredients. The water broth described above is also an ingredient in the mask. The other ingredients are added as preservatives, solvents, and viscosity control agents and are not part of the active ingredients, but rather are a delivery system. Effective formulations arise by selecting elements from each category of ingredients. The delivery system components are but one, albeit the best mode, of potential delivery systems. The best mode used all of the listed ingredients in the amounts indicated. The two-part system of mixing serum and powdered mask ingredients are blended just prior to use.

### Mask Mixing serum

### Herbs

2 fluid ounces Aloe Vera gel

### Fruits

3 fluid ounces Noni Juice

### Butters and Oils

1 fluid ounces Shea Butter
1 fluid ounces Murmuru Butter
1 fluid ounces Cupuacu Butter
2 fluid ounces Capryllic/Capric Triglyceride
5 fluid ounces Rice Bran Oil
5 fluid ounces Argan Oil
3 fluid ounces Camellia Oil
3 fluid ounces Rosehip Seed Oil
2 fluid ounces Dilo Nut Oil
3 fluid ounces Pomegranate Oil
3 fluid ounces Red Raspberry Seed Oil

### Teas

2 fluid ounces green tea liquid extract
4 fluid ounces white tea liquid extract
3 fluid ounces damiana tea (mexico) liquid extract

### Other Additives

1 fluid ounces Xanthan Gum
10 fluid ounces the water broth
7 fluid ounces Squalane from olives
10 fluid ounces Hyaluronic Acid
2 fluid ounces Soy Lecithin
2 fluid ounces Vegetal Glycerol
1 fluid ounces Coco Glucoside
1 fluid ounces Sucrose Stearate
1 fluid ounce 35% hydrogen peroxide

### Dry Mask Ingredients

### Algae and Seaweeds

10 ounces Red Seaweed (Callophycus Serratus)
10 ounce Bladderwrack (Fucus Vesiculosus)
10 ounces Spirulina Blue Green Algae (microcystis aeruginosa)

### Flowers

6 ounces Roman Chamomile
2 ounce Calendula

### Herbs

1 ounce Birch Bark
2 ounce Comfrey Leaf
2 ounces Lady's Mantle
3 ounces Marshmallow root
3 ounces Gotu Kola

### Fruits

4 ounces Acai Berry
4 ounces HawaiianNoni

### Vegetables

1 ounce Chard
1 ounce Spinach
1 ounce Kale

### Other Ingredients

4 ounces Powdered Goat's Milk
4 ounces Powdered Coconut Milk

The mixing serum ingredients are blended together then the “Other Additives” are added. The solution is then treated for 25 minutes by diffusion of output from an ozone generator at 250 mg/hour ozone generation. The hydrogen peroxide is added last. The solution is mixed well and bottled.

The powdered ingredients are pulverized separately into a triple milled particle powder. They are then blended together and sifted approximately 20 times until all ingredients are mixed well and a uniform particle size is obtained to produce the powdered mask base.

### Cream

The final treatment step after removal of the mask is application of a sealing and moisturizing cream. As for the other treatment formulations, the cream is prepared of selections from primary categories noted for particular efficacy or known effective ingredients. The cream provides a longer term application of the effective ingredients in yet another formulation matrix. Effective preparations were made using selections from each of the categories of ingredients defined below. The best mode used all of the indicated ingredients in the proportions listed.

### Algae and Seaweeds

### 5 Ounces Red Seaweed (Callophycus Serratus)

### 2 Ounces Atlantic Sea Kelp (Luminaria Digitata)

### 2 Ounces Bladderwrack (Fucus Vesiculosus)

### Teas

2 fluid ounces white tea extract
1 fluid ounce oolong tea extract
2 fluid ounces green emperor tea extract
3 fluid ounces Damiana tea extract

### Herbs

1 fluid ounce olive leaf extract
1 fluid ounce fennel seed extract
1 fluid ounce rice bran extract
1 ounce licorice root extract
1 fluid ounce Rose Damascens extract
1 fluid ounce Indian Gooseberry extract
2 fluid ounces Gotu Kola extract
2 fluid ounces Gingko Biloba extract
2 fluid ounces lady's mantle extract
1 fluid ounce Comfrey leaf extract
1 fluid ounce Burdock root extract
1 fluid ounce rosemary extract
10 fluid ounces aloe vera gel

### Butters and Oils

1 fluid ounce Cupuacu Butter
1 fluid ounce Murmuru Butter
1 fluid ounce Shea Butter
3 fluid ounces Argan Oil
2 fluid ounces rosehip seed oil
2 fluid ounces evening primrose oil
1 fluid ounce Dilo nut oil
1 fluid ounce Moluccana Oil
1 fluid ounce palmarosa oil
1 fluid ounce rosewood oil
2 fluid ounces safflower seed oil
1 fluid ounce sunflower seed oil
1 fluid ounce pumpkin seed oil
1 fluid ounce Grapeseed oil
1 fluid ounce soybean oil
1 fluid ounce Caprylic/Capric Triglyceride

### Flowers

1 fluid ounce Lavender extract
1 fluid ounce Lotus extract
1 fluid ounce White Iris extract
1 fluid ounce Sea Buckthorn extract
2 fluid ounces Roman Chamomile extract
1 fluid ounce Elderberry blossom extract 1 fluid ounce Calendula extract

### Fruits

3 fluid ounces Hawaiian Noni Juice
3 fluid ounces Acai Berry juice
3 fluid ounces pomegranate juice
2 fluid ounces grape juice

### Vegetables

1 fluid ounce Tomato extract
1 fluid ounce Cucumber extract
1 fluid ounce Kale extract
1 fluid ounce Spinach extract
1 fluid ounce Chard extract

### Other Additives

60 Fluid ounces of the water broth
2 fluid ounces of Virgin organic coconut milk
1 fluid ounce Vegetal Glycerin
1 fluid ounce Sorbitan
1 fluid ounce Glycerol Monostearate
1 fluid ounce Vitamin A Acetate
1 fluid ounce Vitamin E Acetate
1 ounce Zinc Stearate
1 ounce Vitamin B3
1 ounce Vitamin B5
2 ounces lycopene
2 ounces Lysine
2 ounces Proline
2 ounces Serine
1 ounce lactic acid
2 ounces malic acid
1 fluid ounce soy lecithin
1 ounce Acetyl hexapeptide
1 fluid ounce hydrogen Peroxide 35%

The selections from each category of algae and seaweeds, teas, herbs, butters and oils, flowers, fruits and vegetables are blended together. The ingredients are heated just sufficiently to allow thorough mixing without cooking or boiling the constituents. The “Other Additives” are then added. The solution is then treated for 25 minutes by diffusion of output from an ozone generator at 250 mg/hour ozone generation. The hydrogen peroxide is added last. The solution is again mixed well and bottled.

### Treatment Procedure

The treatment procedure comprises application of each of the above described formulations in the order of cleansing with the water broth, activating with both the water broth and the activating serum, treatment with the mask formulation that entraps active ingredients against the affected skin and finally sealing and protecting the treated area with the cream. The affected area is first washed using the water broth. Activating serum is then applied to the skin. The water broth solution is put into the dispensing cup of aerosol generating device such as sold under the brand name Dermoxyzone Nebulizing Nanoparticle Infuser™, which infuses the nanoparticle sizes of the ozonated water broth into the epidermal layer. Absorption is optimized due to the activating serum's ability to pull moisture into the epidermis in greater amounts than could be absorbed without application of the activating serum. The Gel is not completely removed before applying the mask. The gel is wiped down with a cotton pad, leaving a residue on the skin. The mask is then applied over the residual gel covered area of the skin. The mixing serum and the powdered mask base are blended in a ratio of one to one, by volume, to form the mask just prior to use. For the treatment of one cheek for example, one tablespoon each of the powder and mixing serum are typically used. The mask is applied on the skin over the residual activating gel. An ozonated water solution comprising ozone sterilized water and 3% hydrogen peroxide is applied four drops at a time over the mask. Cotton pads are then placed on top of the mask. Slight pressure is applied. When skin begins to tingle, six more drops of the water peroxide solution are added over the cotton pads. If area being treated is for sun damage, the cotton pads are lifted and the additional solution is placed directly onto the skin covered with the mask; the cotton pads are then replaced to cover the mask. Treatment is continued for 4 to 5 minutes. The mask is then wiped with cotton pads and the skin is rinsed well with tepid water until the mask is completely removed. The cream is applied as the last step. For added sun protection, pure zinc oxide powder, such as that sold under the brand name Purleyzinc Micronized Zinc Powder from Hope Aesthetics, Inc. is lightly applied to the skin over the top of the cream.

### EXAMPLE 1

A 46 year old female with basal cell carcinoma and pre-cancerous inflammation was treated conventionally by a medical practitioner using liquid nitrogen to remove cancerous cells. Treatment produced scarring and cancerous tissue reappeared within a few months of treatment. Treatment with a best mode solution of the water broth as a mist reduced inflammation but even with continued treatment cancer and pre-cancerous inflammation recurred. Treatment with a non-ozonated version of the cream alone had little or no effect on inflammation. Treatment with an ozonated cream of the best mode formulation show slight efficacy through reduced inflammation. Treatment with combination of water broth as a mist, then activating serum and finally the best mode cream formulation produced immediate benefit, which could be maintained by frequent, at least weekly, use. Treatment with the series of best mode ozonated formulations as listed above comprising treatment with water broth mist, immediately followed by topical application of the activating serum, then the mask and finally cream produced immediate benefit. Using the combination of formulations in a multiple step treatment regime produced profound reduction in inflammation and elimination of the basal cell carcinoma in a matter of a few weeks. Results were sufficiently profound that the treatment frequency, initially weekly was reduced in increments to the point that now inflammation is controlled with just quarterly treatments.

### EXAMPLE 2

A 55 year old male suffered from chronic rosacea. Medical practitioner proscribed regular use of a steroidal cortisone anti-inflammatory cream. Topical 1% cortisone application if repeated at least weekly controlled most inflammation outbreaks. Patient was concerned about side effects of long term cortisone application. Treatment with the best mode formulations of mist and cream produced immediate improvement. Improvement however could not be maintained even with regular treatment several times weekly. Inflammation returned after the initial improvement. Combination of the mist and cream with occasional topical cortisone treatment controlled inflammation for period of several weeks. Requirement for cortisone treatment have been reduced from weekly to every other week.

### EXAMPLE 3

Four women patients of age 25 to 55 were suffering from melasma, a darkening of the skin associated with hormonal changes from pregnancy, use of birth control pills and hormonal replacement therapies during menopause. The patients had been treated by physicians who proscribed chemical peels and use of topical steroidal creams with limited success. Patients were then treated with the best mode treatment procedure described above. Discoloration and mottling was reduced, with three weekly treatments, in all cases by at least 90% on a visual scale where 0% was the condition of the patient prior to the treatment of this invention and 100% would be the skin judged to have no melasma discoloration. Two of the patients showed a 50% improvement after just a single treatment.

Though the several embodiments and descriptions presented in this document apply to a specific set of pharmaceutically active compounds, the principles involved have application in many situations where skin treatment medications are to be topically applied. There are other applications of the formulations and procedures including combinations of the techniques with other medication steps which are not illustrated above but which but which are clearly within the scope and spirit of the present invention.

### CONCLUSIONS

A series of natural herbal-based ozonated formulations and treatment steps effective in the treatment and preventive maintenance of inflammatory skin conditions is described. The formulations provide a novel approach to selection and preparation of medicated formulations where a cocktail approach is appropriate and a logical series of delivery matrices and processes are used to deliver the active components to the affected region or zone of the anatomy.

I claim:

1-10. (canceled)

11. A water based topical skin treatment formulation comprising a mineral water based extract of teas, extract of algaes and seaweeds, extract of herbs, extract of flowers, extract of fruits, extract of vegetables, and, arbutin, wherein:
a) said teas are at least one selected from the group consisting of white tea, green tea, Rooibos tea, Damiana tea, and Oolong tea, and
b) said algae and seaweeds are at least on at least one selected from the group consisting of Spirulina Blue Green Algae (microcystis aeruginosa), Sea Kelp (Laminaria Hyperborean), Atlantic Sea Kelp (Luminaria Digitata), Bladderwrack (Fucus Vesiculosus), Red Seaweed (Callophycus Serratus), Knotted wrack (Ascophyllum Nodosom), Brown Algae (Phaeophyta), Sea Parsley (Levisticum officinale), Seal Lettuce (Ulva), and
c) said herbs are at least one selected from the group consisting of St. John's Wort, Indian Gooseberry, slippery elm, corkwood tree, chickweed, Nopal, Gotu Kola, Black willow, olive leaf, grape leaf, Gingko Biloba, rosemary, usnea, Siegesbeckia Orientalis, rosehip seed, flax seed, white ginger, Licorice Root, White Birch Bark, Argan, Duboisia, Horse Chestnut, Rice Bran, Camellia, Dilo nut, Calendula, Comfrey, Lady's Mantle, Marshmallow root, evening primrose, Moluccana, Palmarosa, rosewood, safflower, sunflower seed, fennel seed, rosemary, aloe vera, Burdock Root, and
d) said flowers are at least one selected from the group consisting of Roman Chamomile, hibiscus, lavender, borage, camellia, sunflower, Calendula, Elderberry Blossom, Lotus, Linden, Lichen, Sea Buckthorn, white Iris, rose damascens, and Comfrey, and
e) said fruits are at least one selected from the group consisting of Apple, persimmon, Grapes, papaya, Italian Melon, pineapple, pomegranate, black cherry, red raspberry, blueberry, Kiwano Melon, cranberry, Hawaiian noni, Brazilian Acai berry, pipano melon, starfruit, Prickly Pear, and Brazilian melon, and
f) said vegetables are at least one selected from the group consisting of carrot, spinach, kale, chard, tomato, and cucumber.

12. The water based topical skin treatment formulation of claim 11 further comprising hydrogen peroxide and wherein said formulation is treated with ozone.

13. A topical skin treatment serum comprising tea extracts, algae and seaweed extracts, herb extracts, butters and oils, flower extracts, fruit extracts, arbutin, and, hyaluronic acid, wherein:
a) said teas are at least one selected from the group consisting of white tea, green tea, Rooibos tea, Damiana tea, and Oolong tea, and
b) said algae and seaweeds are at least on at least one selected from the group consisting of Spirulina Blue Green Algae (microcystis aeruginosa), Sea Kelp (Laminaria Hyperborean), Atlantic Sea Kelp (Luminaria Digitata), Bladderwrack (Fucus Vesiculosus), Red Seaweed (Callophycus Serratus), Knotted wrack (Ascophyllum Nodosom), Brown Algae (Phaeophyta), Sea Parsley (Levisticum officinale), Seal Lettuce (Ulva), and
c) said herbs are at least one selected from the group consisting of St. John's Wort, Indian Gooseberry, slippery elm, corkwood tree, chickweed, Nopal, Gotu Kola, Black willow, olive leaf, grape leaf, Gingko Biloba, rosemary, usnea, Siegesbeckia Orientalis, rosehip seed, flax seed, white ginger, Licorice Root, White Birch Bark, Argan, Duboisia, Horse Chestnut, Rice Bran, Camellia, Dilo nut, Calendula, Comfrey, Lady's Mantle, Marshmallow root, evening primrose, Moluccana, Palmarosa, rosewood, safflower, sunflower seed, fennel seed, rosemary, aloe vera, Burdock Root, and
d) said flowers are at least one selected from the group consisting of Roman Chamomile, hibiscus, lavender, borage, camellia, sunflower, Calendula, Elderberry Blossom, Lotus, Linden, Lichen, Sea Buckthorn, white Iris, rose damascens, and Comfrey, and
e) said fruits are at least one selected from the group consisting of Apple, persimmon, Grapes, papaya, Italian Melon, pineapple, pomegranate, black cherry, red raspberry, blueberry, Kiwano Melon, cranberry, Hawaiian noni, Brazilian Acai berry, pipano melon, starfruit, Prickly Pear, and Brazilian melon, and
f) said vegetables are at least one selected from the group consisting of carrot, spinach, kale, chard, tomato, and cucumber, and
g) said butters and oils are at least one selected from the group consisting of Shea
Butter, Murmuru Butter, Cupuacu Butter, Capryllic/Capric Triglyceride, Shea Butter, Rice Bran Oil, Argan Oil, Camellia Oil, Rosehip Seed Oil, Dilo Nut Oil, Pomegranate Oil, Red Raspberry Seed Oil, evening primrose oil, Moluccana Oil, palmarosa oil, rosewood oil, safflower seed oil, sunflower seed oil, pumpkin seed oil, Grapeseed oil and soybean oil.

14. The topical skin treatment serum of claim 13 further comprising hydrogen peroxide and wherein said serum is treated with ozone.

15. A mask treatment formulation comprising a dry ingredient formulation and a mixing serum formulation wherein said dry ingredient formulation comprises powdered goat's milk, powdered coconut milk and:
a) at least one tea selected from the group consisting of white tea, green tea, Rooibos tea, Damiana tea, and Oolong tea, and
b) at least one algae and seaweed selected from the group consisting of Spirulina Blue Green Algae (microcystis aeruginosa), Sea Kelp (Laminaria Hyperborean), Atlantic Sea Kelp (Luminaria Digitata), Bladderwrack (Fucus Vesiculosus), Red Seaweed (Callophycus Serratus), Knotted wrack (Ascophyllum Nodosom), Brown Algae (Phaeophyta), Sea Parsley (Levisticum officinale), Seal Lettuce (Ulva), and
c) at least one herb selected from the group consisting of St. John's Wort, Indian Gooseberry, slippery elm, corkwood tree, chickweed, Nopal, Gotu Kola, Black willow, olive leaf, grape leaf, Gingko Biloba, rosemary, usnea, Siegesbeckia Orientalis, rosehip seed, flax seed, white ginger, Licorice Root, White Birch Bark, Argan, Duboisia, Horse Chestnut, Rice Bran, Camellia, Dilo nut, Calendula, Comfrey, Lady's Mantle, Marshmallow root, evening primrose, Moluccana, Palmarosa, rosewood, safflower, sunflower seed, fennel seed, rosemary, aloe vera, Burdock Root, and
d) at least one flower selected from the group consisting of Roman Chamomile, hibiscus, lavender, borage, camellia, sunflower, Calendula, Elderberry Blossom, Lotus, Linden, Lichen, Sea Buckthorn, white Iris, rose damascens, and Comfrey, and
e) at least one fruit selected from the group consisting of Apple, persimmon, Grapes, papaya, Italian Melon, pineapple, pomegranate, black cherry, red raspberry, blueberry, Kiwano Melon, cranberry, Hawaiian noni, Brazilian Acai berry, pipano melon, starfruit, Prickly Pear, and Brazilian melon, and
f) at least one vegetable selected from the group consisting of carrot, spinach, kale, chard, tomato, and cucumber, and
wherein said mixing serum comprises hyaluronic acid and:
a) at least one herb extract selected from the group consisting of St. John's Wort, Indian Gooseberry, slippery elm, corkwood tree, chickweed, Nopal, Gotu Kola, Black willow, olive leaf, grape leaf, Gingko Biloba, rosemary, usnea, Siegesbeckia Orientalis, rosehip seed, flax seed, white ginger, Licorice Root, White Birch Bark, Argan, Duboisia, Horse Chestnut, Rice Bran, Camellia, Dilo nut, Calendula, Comfrey, Lady's Mantle, Marshmallow root, evening primrose, Moluccana, Palmarosa, rosewood, safflower, sunflower seed, fennel seed, rosemary, aloe vera, Burdock Root, and
b) at least one fruit extract selected from the group consisting of Apple, persimmon, Grapes, papaya, Italian Melon, pineapple, pomegranate, black cherry, red raspberry, blueberry, Kiwano Melon, cranberry, Hawaiian noni, Brazilian Acai berry, pipano melon, starfruit, Prickly Pear, and Brazilian melon, and
c) at least one butter and oil selected from the group consisting of Shea Butter, Murmuru Butter, Cupuacu Butter, Capryllic/Capric Triglyceride, Shea Butter, Rice Bran Oil, Argan Oil, Camellia Oil, Rosehip Seed Oil, Dilo Nut Oil, Pomegranate Oil, Red Raspberry Seed Oil, evening primrose oil, Moluccana Oil, palmarosa oil, rosewood oil, safflower seed oil, sunflower seed oil, pumpkin seed oil, Grapeseed oil and soybean oil, and
d) at least one tea extract selected from the group consisting of white tea, green tea, Rooibos tea, Damiana tea, and Oolong tea.

16. The mask treatment formulation of claim 15 said mixing serum formulation further comprising hydrogen peroxide and wherein said mixing serum formulation is treated with ozone.

17. A topical skin cream comprising
a) at least one tea extract selected from the group consisting of white tea, green tea, Rooibos tea, Damiana tea, and Oolong tea, and
b) at least one algae and seaweed extract selected from the group consisting of Spirulina Blue Green Algae (microcystis aeruginosa), Sea Kelp (Laminaria Hyperborean), Atlantic Sea Kelp (Luminaria Digitata), Bladderwrack (Fucus Vesiculosus), Red Seaweed (Callophycus Serratus), Knotted wrack (Ascophyllum Nodosom), Brown Algae (Phaeophyta), Sea Parsley (Levisticum officinale), Seal Lettuce (Ulva), and
c) at least one herb extract selected from the group consisting of St. John's Wort, Indian Gooseberry, slippery elm, corkwood tree, chickweed, Nopal, Gotu Kola, Black willow, olive leaf, grape leaf, Gingko Biloba, rosemary, usnea, Siegesbeckia Orientalis, rosehip seed, flax seed, white ginger, Licorice Root, White Birch Bark, Argan, Duboisia, Horse Chestnut, Rice Bran, Camellia, Dilo nut, Calendula, Comfrey, Lady's Mantle, Marshmallow root, evening primrose, Moluccana, Palmarosa, rosewood, safflower, sunflower seed, fennel seed, rosemary, aloe vera, Burdock Root, and
d) at least one butter and oil selected from the group consisting of Shea Butter, Murmuru Butter, Cupuacu Butter, Capryllic/Capric Triglyceride, Shea Butter, Rice Bran Oil, Argan Oil, Camellia Oil, Rosehip Seed Oil, Dilo Nut Oil, Pomegranate Oil, Red Raspberry Seed Oil, evening primrose oil, Moluccana Oil, palmarosa oil, rosewood oil, safflower seed oil, sunflower seed oil, pumpkin seed oil, Grapeseed oil and soybean oil, and
e) at least one flower extract selected from the group consisting of Roman Chamomile, hibiscus, lavender, borage, camellia, sunflower, Calendula, Elderberry Blossom, Lotus, Linden, Lichen, Sea Buckthorn, white Iris, rose damascens, and Comfrey, and
f) at least one fruit extract selected from the group consisting of Apple, persimmon, Grapes, papaya, Italian Melon, pineapple, pomegranate, black cherry, red raspberry, blueberry, Kiwano Melon, cranberry, Hawaiian noni, Brazilian Acai berry, pipano melon, starfruit, Prickly Pear, and Brazilian melon, and
g) at least one vegetable extract selected from the group consisting of carrot, spinach, kale, chard, tomato, and cucumber.

18. The topical skin cream of claim 17 further comprising hydrogen peroxide and wherein said skin cream is treated with ozone.

19. A water based topical skin treatment formulation comprising a mineral water based extract of teas, extract of callophycus serratus, extract of herbs, extract of flowers, extract of fruits, extract of vegetables, and, arbutin, wherein:
a) said teas are at least one selected from the group consisting of white tea, green tea, Rooibos tea, Damiana tea, and Oolong tea, and
b) said herbs are at least one selected from the group consisting of St. John's Wort, Indian Gooseberry, slippery elm, corkwood tree, chickweed, Nopal, Gotu Kola, Black willow, olive leaf, grape leaf, Gingko Biloba, rosemary, usnea, Siegesbeckia Orientalis, rosehip seed, flax seed, white ginger, Licorice Root, White Birch Bark, Argan, Duboisia, Horse Chestnut, Rice Bran, Camellia, Dilo nut, Calendula, Comfrey, Lady's Mantle, Marshmallow root, evening primrose, Moluccana, Palmarosa, rosewood, safflower, sunflower seed, fennel seed, rosemary, aloe vera, Burdock Root, and
c) said flowers are at least one selected from the group consisting of Roman Chamomile, hibiscus, lavender, borage, camellia, sunflower, Calendula, Elderberry Blossom, Lotus, Linden, Lichen, Sea Buckthorn, white Iris, rose damascens, and Comfrey, and
d) said fruits are at least one selected from the group consisting of Apple, persimmon, Grapes, papaya, Italian Melon, pineapple, pomegranate, black cherry, red raspberry, blueberry, Kiwano Melon, cranberry, Hawaiian noni, Brazilian Acai berry, pipano melon, starfruit, Prickly Pear, and Brazilian melon, and
e) said vegetables are at least one selected from the group consisting of carrot, spinach, kale, chard, tomato, and cucumber.

20. The water based topical skin treatment formulation of claim 19 further comprising hydrogen peroxide and wherein said formulation is treated with ozone.

21. A topical skin treatment serum comprising tea extracts, extract of callophycus serratus, herb extracts, butters and oils, flower extracts, fruit extracts, arbutin, and, hyaluronic acid, wherein:
a) said teas are at least one selected from the group consisting of white tea, green tea, Rooibos tea, Damiana tea, and Oolong tea, and
b) said herbs are at least one selected from the group consisting of St. John's Wort, Indian Gooseberry, slippery elm, corkwood tree, chickweed, Nopal, Gotu Kola, Black willow, olive leaf, grape leaf, Gingko Biloba, rosemary, usnea, Siegesbeckia Orientalis, rosehip seed, flax seed, white ginger, Licorice Root, White Birch Bark, Argan, Duboisia, Horse Chestnut, Rice Bran, Camellia, Dilo nut, Calendula, Comfrey, Lady's Mantle, Marshmallow root, evening primrose, Moluccana, Palmarosa, rosewood, safflower, sunflower seed, fennel seed, rosemary, aloe vera, Burdock Root, and
c) said flowers are at least one selected from the group consisting of Roman Chamomile, hibiscus, lavender, borage, camellia, sunflower, Calendula, Elderberry Blossom, Lotus, Linden, Lichen, Sea Buckthorn, white Iris, rose damascens, and Comfrey, and
d) said fruits are at least one selected from the group consisting of Apple, persimmon, Grapes, papaya, Italian Melon, pineapple, pomegranate, black cherry, red raspberry, blueberry, Kiwano Melon, cranberry, Hawaiian noni, Brazilian Acai berry, pipano melon, starfruit, Prickly Pear, and Brazilian melon, and
e) said vegetables are at least one selected from the group consisting of carrot, spinach, kale, chard, tomato, and cucumber, and
f) said butters and oils are at least one selected from the group consisting of Shea Butter, Murmuru Butter, Cupuacu Butter, Capryllic/Capric Triglyceride, Shea Butter, Rice Bran Oil, Argan Oil, Camellia Oil, Rosehip Seed Oil, Dilo Nut Oil, Pomegranate Oil, Red Raspberry Seed Oil, evening primrose oil, Moluccana Oil, palmarosa oil, rosewood oil, safflower seed oil, sunflower seed oil, pumpkin seed oil, Grapeseed oil and soybean oil.

22. The topical skin treatment serum of claim 21 further comprising hydrogen peroxide and wherein said serum is treated with ozone.

23. A mask treatment formulation comprising a dry ingredient formulation and a mixing serum formulation wherein said dry ingredient formulation comprises powdered goat's milk, powdered coconut milk, extract of callophycus serratus, and:
a) at least one tea selected from the group consisting of white tea, green tea, Rooibos tea, Damiana tea, and Oolong tea, and
b) at least one herb selected from the group consisting of St. John's Wort, Indian Gooseberry, slippery elm, corkwood tree, chickweed, Nopal, Gotu Kola, Black willow, olive leaf, grape leaf, Gingko Biloba, rosemary, usnea, Siegesbeckia Orientalis, rosehip seed, flax seed, white ginger, Licorice Root, White Birch Bark, Argan, Duboisia, Horse Chestnut, Rice Bran, Camellia, Dilo nut, Calendula, Comfrey, Lady's Mantle, Marshmallow root, evening primrose, Moluccana, Palmarosa, rosewood, safflower, sunflower seed, fennel seed, rosemary, aloe vera, Burdock Root, and
c) at least one flower selected from the group consisting of Roman Chamomile, hibiscus, lavender, borage, camellia, sunflower, Calendula, Elderberry Blossom, Lotus, Linden, Lichen, Sea Buckthorn, white Iris, rose damascens, and Comfrey, and
d) at least one fruit selected from the group consisting of Apple, persimmon, Grapes, papaya, Italian Melon, pineapple, pomegranate, black cherry, red raspberry, blueberry, Kiwano Melon, cranberry, Hawaiian noni, Brazilian Acai berry, pipano melon, starfruit, Prickly Pear, and Brazilian melon, and
e) at least one vegetable selected from the group consisting of carrot, spinach, kale, chard, tomato, and cucumber, and
wherein said mixing serum comprises hyaluronic acid and:
f) at least one herb extract selected from the group consisting of St. John's Wort, Indian Gooseberry, slippery elm, corkwood tree, chickweed, Nopal, Gotu Kola, Black willow, olive leaf, grape leaf, Gingko Biloba, rosemary, usnea, Siegesbeckia Orientalis, rosehip seed, flax seed, white ginger, Licorice Root, White Birch Bark, Argan, Duboisia, Horse Chestnut, Rice Bran, Camellia, Dilo nut, Calendula, Comfrey, Lady's Mantle, Marshmallow root, evening primrose, Moluccana, Palmarosa, rosewood, safflower, sunflower seed, fennel seed, rosemary, aloe vera, Burdock Root, and
g) at least one fruit extract selected from the group consisting of Apple, persimmon, Grapes, papaya, Italian Melon, pineapple, pomegranate, black cherry, red raspberry, blueberry, Kiwano Melon, cranberry, Hawaiian noni, Brazilian Acai berry, pipano melon, starfruit, Prickly Pear, and Brazilian melon, and
h) at least one butter and oil selected from the group consisting of Shea Butter, Murmuru Butter, Cupuacu Butter, Capryllic/Capric Triglyceride, Shea Butter, Rice Bran Oil, Argan Oil, Camellia Oil, Rosehip Seed Oil, Dilo Nut Oil, Pomegranate Oil, Red Raspberry Seed Oil, evening primrose oil, Moluccana Oil, palmarosa oil, rosewood oil, safflower seed oil, sunflower seed oil, pumpkin seed oil, Grapeseed oil and soybean oil, and
i) at least one tea extract selected from the group consisting of white tea, green tea, Rooibos tea, Damiana tea, and Oolong tea.

24. The mask treatment formulation of claim 23 said mixing serum formulation further comprising hydrogen peroxide and wherein said mixing serum formulation is treated with ozone.

25. A topical skin cream comprising:
a) at least one tea extract selected from the group consisting of white tea, green tea, Rooibos tea, Damiana tea, and Oolong tea, and
b) extracts of Callophycus Serratus, and
c) at least one herb extract selected from the group consisting of St. John's Wort, Indian Gooseberry, slippery elm, corkwood tree, chickweed, Nopal, Gotu Kola, Black willow, olive leaf, grape leaf, Gingko Biloba, rosemary, usnea, Siegesbeckia Orientalis, rosehip seed, flax seed, white ginger, Licorice Root, White Birch Bark, Argan, Duboisia, Horse Chestnut, Rice Bran, Camellia, Dilo nut, Calendula, Comfrey, Lady's Mantle, Marshmallow root, evening primrose, Moluccana, Palmarosa, rosewood, safflower, sunflower seed, fennel seed, rosemary, aloe vera, Burdock Root, and
d) at least one butter and oil selected from the group consisting of Shea Butter, Murmuru Butter, Cupuacu Butter, Capryllic/Capric Triglyceride, Shea Butter, Rice Bran Oil, Argan Oil, Camellia Oil, Rosehip Seed Oil, Dilo Nut Oil, Pomegranate Oil, Red Raspberry Seed Oil, evening primrose oil, Moluccana Oil, palmarosa oil, rosewood oil, safflower seed oil, sunflower seed oil, pumpkin seed oil, Grapeseed oil and soybean oil, and
e) at least one flower extract selected from the group consisting of Roman Chamomile, hibiscus, lavender, borage, camellia, sunflower, Calendula, Elderberry Blossom, Lotus, Linden, Lichen, Sea Buckthorn, white Iris, rose damascens, and Comfrey, and
f) at least one fruit extract selected from the group consisting of Apple, persimmon, Grapes, papaya, Italian Melon, pineapple, pomegranate, black cherry, red raspberry, blueberry, Kiwano Melon, cranberry, Hawaiian noni, Brazilian Acai berry, pipano melon, starfruit, Prickly Pear, and Brazilian melon, and
g) at least one vegetable extract selected from the group consisting of carrot, spinach, kale, chard, tomato, and cucumber.

26. The topical skin cream of claim 25 further comprising hydrogen peroxide and wherein said skin cream is treated with ozone.
